(19) United States
(12) Patent Application Publication
(10) Pub. No.: US 2025/0120588 A1
(43) Pub. Date: Apr. 17, 2025

(54) Optical Measurement Apparatus

(71) Applicant: University of Strathclyde, Glasgow (GB)

(72) Inventors: Mario E. GIARDINI, Glasgow (GB); Ian COGHILL, Glasgow (GB); Kristy JORDAN, Glasgow (GB); Richard BLACK, Glasgow (GB)

(21) Appl. No.: 18/723,130

(22) PCT Filed: Nov. 28, 2022
(86) PCT No.: PCT/GB2022/053003

§ 371 Date: Jun. 21, 2024

(30) Foreign Application Priority Data
Dec. 21, 2021 (GB) 2118643.2

(51) Int. Cl. A61B 3/14

(52) U.S. Cl. CPC A61B 3/145

(57) ABSTRACT

An eye imaging system including: an illumination device configured to illuminate a structure of an eye with a patterned or otherwise textured image; one or more image collection devices wherein; the one or more image collection devices are arranged to capture one or more images of the structure of the eye from one of a plurality of physical locations and/or from different perspectives, e.g. the capture images of the structure of the eye whilst the structure of the eye is illuminated by the patterned or otherwise textured image.

### FIELD

The present disclosure relates to an optical measurement apparatus, such as an eye imaging system, and associated method for capturing images of structures of the eye using the eye imaging system which allows for a three-dimensional image to be generated from those images.

### BACKGROUND

Imaging of the eye, and particularly three-dimensional mapping of various internal and external structures of the eye is an important tool in the field of ophthalmology. Examples of such structures of the eye include the optic nerve head (the point where the optic nerve joins the retina) and the cornea (the clear outer layer at the front of the eye). An accurate mapping of such structures can be used to identify the structures, and prepare for procedures related to important pathologies of the eye.

For example, knowing the three-dimensional shape of the optic nerve head may allow identification of the presence of glaucoma, which affects the size and the amount of recess of the optic nerve head. Currently estimating the shape of the optic nerve head is normally done subjectively, by direct observation of the optic nerve head through a slit lamp (a binocular microscope that, with the assistance of a lens interposed between microscope and eye, can visualise the retina) or by using a stereoscopic retinal camera, that captures two images of the eye from moderately different angles, thus allowing the operator to visualise depth by observing the two images simultaneously, one with each eye. An alternative technology is Optical Coherence Tomography (OCT) which unlike direct observation of the disc, is objective. However, traditionally OCT machines are bulky, heavy, and also extremely expensive.

As another example, when planning for corneal transplants, the full cornea is mapped, allowing steepness measurements to be estimated. These can be used to calculate the corneal refractive power and/or to plan the surgery for the desired optical outcome. Traditional corneal topography equipment can only measure the shape of the cornea close to the corneal centre. As a map of the full cornea is required, the current standard technique to access the shape of the peripheral regions consists of taking a mould of the cornea using a moulding compound, and measuring the mould. This is both complex, and painful for the patient.

Other examples for which no simple and/or cost-effective tool for suitable three-dimensional mapping exists include the identification of retinal detachments and choroidal melanomas. Choroidal melanomas appear as dark spots on the retina differentiated from choroidal nevi in that they are raised rather than flat relative to the surface of the retina.

However, the above are merely examples of applications for which an eye imaging system could be useful and optical measurement apparatus that can image structures of the eye could be applied across a range of other applications, such as research, monitoring of the eye, and the like.

It would be beneficial to have a simple, cost-effective system for producing a high spatial resolution three-dimensional image of internal and external structures of the eye, causing minimal discomfort to the patient and substantially impervious to eye movements.

### SUMMARY

Various aspects of the present invention are defined in the independent claims. Some preferred features are defined in the dependent claims.

According to a first aspect of the present disclosure is an eye imaging system comprising an illumination device configured to illuminate a structure of an eye with a patterned or otherwise textured image. The eye imaging system may further comprise a one or more image collection devices, wherein the one or more image collection devices may be arranged to capture one or more images of the structure of the eye from one of a plurality of physical locations and/or perspectives.

The eye imaging system may comprise a slit lamp. The illumination device may be comprised in the slit lamp.

The eye imaging system may optionally comprise a beam splitter. The beam splitter may be provided in either or both optical pathways between: (i) the illumination device and the eye; and/or (ii) between the one or more image collection devices and the eye. The beam splitter may be arranged to reflect light from the illumination device towards the eye. The beam splitter may be arranged to transmit the light reflected back from the eye to the one or more image collection devices.

The eye imaging system (e.g. the slit lamp) may comprise a routing system for selectively providing reflections of the patterned or otherwise textured image, or to induce a pattern in fluorescence or photoluminescence. The fluorescence or photo-luminescence may be produced by fluorescent or photo luminescent material applied to the eye. The fluorescence or photoluminescence may be either auto-fluorescence or fluorescence of an appropriate contrast agent, from the structure of the eye to the different physical locations and/or with the different perspectives. The routing system may be or comprise a biomicroscope or binocular system, a further beam splitter, a prism, a plurality of mirrors or a multi-facetted mirror, or a movable mirror, or a movable aperture, or the like. The eye imaging system may comprise two or more apertures, such as eyepieces or the like, which may be provided at the different locations. The routing system may be configured to provide the reflections of the patterned or otherwise textured image from the structure of the eye from different perspectives to different apertures. The eye imaging system (e.g. the routing system) may be arranged so that the structure of the eye can be imaged from at least two different perspectives e.g. from two different physical locations, e.g. via the different apertures.

The eye imaging system may be configured to capture a plurality (e.g. two or more) of the images of the structure of the eye substantially simultaneously or one at a time, wherein each image of the plurality of images is captured from a different physical location and/or perspective. The eye imaging system may use a single imaging device that is moved between two or more locations to capture images of the structure of the eye from a different perspective, or comprise two or more image collection devices, where each of the two or more image collection devices may be configured to capture images of the structure of the eye from a different physical location and/or perspective to each of the other imaging devices. Each of the two or more imaging devices may be configured to capture images of the structure of the eye simultaneously. Each of the two or more image collection devices may be configured to receive reflections of the patterned or otherwise textured image of fluorescence images as per above from the structure of the eye, e.g. via respective apertures.

The eye imaging system may comprise a control system for controlling operation of the at least one imaging device. The control system may comprise a synchronising module for synchronising collection of images by the plurality of the image collection devices, e.g. so as to collect images from the at least two different perspectives and/or the two different physical locations substantially simultaneously. The control system may be configured to control the at least one imaging device to collect images of the structure of the eye over time.

The eye imaging system may further comprise an image processing system which may be configured to process the one or more images captured each of the one or more image collection devices, from each of the physical locations and/or perspectives. The image processing system may process the one or more images to produce a three dimensional image, model, or other representation of the structure of the eye illuminated by the illumination device. For example, the image processing system may be configured to produce the 3D model from a plurality of the images, wherein respective images of the plurality of images are captured from different perspectives and/or locations using photogrammetry, such as stereophotogrammetry, geometric techniques, ray tracing, pattern matching, and/or the like.

The patterned or otherwise textured image may comprise a random or pseudorandom pattern, and/or a regular or repeating pattern. The pattern may comprise a fine random dot pattern and/or a set of stripes and/or a grid and/or a geometric or other repeating pattern. The pattern may be a static pattern. The pattern may be a moving pattern or a pattern that otherwise changes in time, e.g. switching on and off, changing in intensity, changing in colour, this all as a whole or in different regions of the pattern.

The illumination device may comprise an illuminator or other light source. The illumination device may comprise an image projector. The illumination device may be integral to the eye imaging system or provided separately. The illumination device may comprise a focussing system, which may be configured for focussing the pattern onto the structure of the eye.

The light source may be configured to have a wavelength which excites autofluorescence from the tissue, or a fluorescent and/or photoluminescent substance, facilitating it to emit light having the same or different wavelength as the light emitted by the light source and incident on the photoluminescent and/or fluorescent substance. The fluorescent and/or photoluminescent substance may be applied to the structure of the eye such that when the structure of the eye is illuminated by light having a wavelength which excites the fluorescence and/or photoluminescence, light is emitted at a wavelength which may be the same or different to the incident light.

The fluorescent and/or photoluminescent substance may comprise a fluorescent dye, which may be fluorescein or another ophthalmic fluorescent dye.

The one or more image collection devices may comprise a light filter. The light filter may be configured to selectively pass the light at the wavelengths emitted by the autofluorescence or the fluorescent and/or photoluminescent substance applied to the structure of the eye, when the structure of the eye is illuminated by the illumination device. The light filter may be configured to filter out substantially all of incident light provided to it, except the light at the wavelengths emitted by the autofluorescence or fluorescent and/or photoluminescent substance applied to the structure of the eye, when the structure of the eye is illuminated by the illumination device.

The eye imaging system may further comprise an indirect ophthalmoscopy lens, which may be positioned in an optical path between the illumination device and/or the structure of the eye and/or the image collection devices. The indirect ophthalmoscopy lens may be positioned in an optical path between the structure of the eye and the beam splitter. The indirect ophthalmoscopy lens may be configured and positioned to work in conjunction with the eye's optics to conjugate the patterned or otherwise textured image to the structure of the eye, e.g. retina. The patterned or otherwise textured image may then be reflected from the structure, e.g. retina, of the eye, or emitted by autofluorescence, fluorescence, or photoluminescence. The indirect ophthalmoscopy lens may be configured to focus the patterned or otherwise textured image reflected from the structure of the eye, or emitted by autofluorescence, fluorescence, or photoluminescence, to a focus point. The focus point may be within a focal length of the biomicroscope. A portion of the focused reflected light may be transmitted through the beam splitter, e.g. to the biomicroscope and/or image collection devices.

According to a second aspect of the present disclosure is a method of capturing one or more images of a structure of an eye using an eye imaging system such as the eye imaging system of the first aspect.

The method comprises illuminating the structure of the eye with a patterned or otherwise textured image using an illumination device and using one or more image collection devices to capture one or more images of the structure of the eye from one of a plurality of physical locations and/or perspectives, and to capture one or more images of the structure of the eye from another one of the plurality of physical locations and/or different perspectives.

The method may comprise imaging the structure of an eye under no external load and/or other factor that changes the shape of the eye, such as the pressure of an object or finger. The method may comprise adding a load or and/or other factor statically and/or dynamically to the structure of the eye before and/or during the capture of one or more images. This may allow the deformation of the structure of the eye in response to such a load and/or other factor to be quantified.

The method may comprise illuminating the structure of the eye with a moving or otherwise changing in time patterned or otherwise textured image or a static image. By collecting multiple images over time whilst projecting a changing image or pattern, the captured images may allow directionality and rate-dependent phenomena (e.g. fluid flow) to be determined, and/or the sensitivity to be increased e.g. through lock-in detection of image intensity modulation, and/or compensation of image artefacts, and/or the image resolution to be enhanced, and/or structured light imaging procedures to be applied, and/or any other of the image enhancements allowed by image processing of time-variant images.

According to a third aspect of the present disclosure is a method for processing one or more images of a structure of an eye captured using an eye imaging system such as the eye imaging system of the first aspect and/or collected using a method of the second aspect. The one or more images may be processed using an image processing system such as the image processing system optionally comprised in the eye imaging system of the first aspect.

The method comprises processing the images to produce a three dimensional image of the structure of the eye illuminated with a patterned or otherwise textured image by an illumination device. The method may comprise producing the 3D image from a plurality of the images, wherein respective images of the plurality of images are captured from different perspectives and/or locations. The producing of the 3D images may comprise processing the plurality of images of the structure of the eye captured from different perspectives and/or locations using photogrammetry, such as stereophotogrammetry, geometric techniques, ray tracing, pattern matching, and/or the like

According to a fourth aspect of the present disclosure is a computer program product configured such that, when executed by an image processing system of an eye imaging system, such as the eye imaging system of the first aspect, causes the eye imaging system to perform the method of the second and/or third aspect.

The computer program product may be embodied on a tangible, non-transient carrier medium, such as but not limited to a memory card, an optical disk or other optical storage, a magnetic disk or other magnetic storage, quantum memory, a memory such as RAM, ROM, a solid state device (SSD) memory, and/or the like. Rather than by using a computer program, part or all of the processing may be embodied in a hardware device, such as a Field-Programmable Gate Array (FPGA), a Complex Programmable Logic Device (CPLD), a manycore vision processing unit, such as e.g. the Intel Movidius Neural Compute Stick, or an inferencing engine, such as e.g. Google Coral or nVidia Jetson Nano.

According to a fifth aspect of the present disclosure is an eye imaging system comprising: an illumination device configured to illuminate a structure of an eye with a patterned or otherwise textured image; and one or more image collection devices arranged to capture one or more images of the structure of the eye; wherein the illumination device is configured to illuminate the structure of the eye with the patterned or otherwise textured image that is moving or otherwise changing in time. One or more image collection devices may be configured to collect multiple images or video of the structure of the eye over time, e.g. whilst the illumination device projects the moving or otherwise changing image or pattern. The collected images may allow directionality and rate-dependent phenomena (e.g. fluid flow) to be determined, and/or the sensitivity to be increased e.g. through lock-in detection of image intensity modulation, and/or compensation of image artefacts, and/or the image resolution to be enhanced, and/or structured light imaging procedures to be applied, and/or any other of the image enhancements allowed by image processing of time-variant images.

Examples of determination of directionality, and rate-dependent phenomena are commonly found in fluid mechanics to infer the velocity of particles in a flow field, namely, in particle image velocimetry and in laser Doppler anemometry, or in non-destructive materials testing to measure deformation. In our system, Digital Image Correlation (DIC) techniques may be used to quantify the displacement of particles or deformation of the illuminated structures within the eye: a series of images of the surface are captured as it undergoes deformation, and the resulting displacement vectors computed using DIC, for example Fast Fourier Transforms and inverses, for example as explained in “Image Correlation for Shape, Motion and Deformation Measurements. Basic Concepts, Theory and Applications”, Sutton, Orteu, Schreier, Springer, 2009

As a more specific example related to measurement of flow, akin to a laser Doppler technique, the illumination device can project a moving pattern of interference fringes (alternating light and dark lines), and the magnitude and direction of motion of particles in the flow field (either already present in the blood, such as red blood cells, or injected in the bloodstream) may be inferred from the intensity modulation of the light scattered from the particles as they pass through the moving fringes at the point of measurement. The particles may be spherical or otherwise, and comprise fluorescent material to improve contrast and signal-to-noise ratio. These principles are well explained in “Laser Systems in Flow Measurement”, Durrani and Greated, Springer, 1977. In the case of a projected image or pattern, the pattern may be made to vary in time by electromechanical means and a mirror, or by purely electronic means, such as using a light modulator or a digital display in the pattern generation optics, to enhance resolution as it is commonly done in the enhancement of the resolution in optical linear or angular absolute encoders (Kohsaka, F., Iino, T., Kazami, K., Nakayama, H., & Ueda, T. (1990). Multiturn absolute encoder using spatial filter. JSME International Journal, 33(1), 94-99)

The patterned image may comprise a regular or repeating pattern. The patterned image may comprise a random or pseudorandom pattern. The patterned or otherwise textured image may comprise a random dot pattern or a set of stripes or a grid.

The eye imaging system may comprise a beam splitter. The beam splitter may be provided in either or both optical pathways between: (i) the illumination device and the eye; and/or (ii) between the one or more image collection devices and the eye. The beam splitter may be arranged to reflect light from the illumination device towards the eye. The beam splitter may be arranged to transmit the light reflected back from the eye to the one or more image collection devices.

The illumination device may comprise an illuminator or other light source or an image projector. The illumination device may be configured to have a wavelength which excites a photoluminescent substance or a fluorescent substance applied to the structure of the eye. The one or more image collection devices may comprise a light filter configured to filter substantially all of the light, except light at the wavelengths emitted by the fluorescent or photoluminescent substance applied to the structure of the eye, when the structure of the eye is illuminated by the illumination device. An indirect ophthalmoscopy lens may be positioned between the illumination device and/or the structure of the eye and/or the image collection devices.

According to a sixth aspect of the present disclosure is a method of capturing one or more images of a structure of an eye using the eye imaging system of the fifth aspect, the method comprising: illuminating the structure of the eye with a moving or otherwise changing patterned or otherwise textured image using the illumination device; and using one or more image collection devices to capture a video or a plurality of images of the structure of the eye over time whilst the illumination device projects the moving or otherwise changing image or pattern.

The method may comprise determining directionality and/or rate-dependent phenomena (e.g. fluid flow) from the video and/or the plurality of images. The method may comprise increasing the sensitivity. The method may comprise using lock-in detection of image intensity modulation, and/or compensation of image artefacts, e.g. to determine the directionality and/or rate-dependent phenomena (e.g. fluid flow) and/or to increasing the sensitivity and/or to enhance the image resolution. The method may comprise applying structured light imaging procedures and/or any other of image enhancements allowed by image processing of time-variant images.

The individual features and/or combinations of features defined above in accordance with any aspect of the present invention or below in relation to any specific embodiment of the invention may be utilised, either separately and individually, alone or in combination with any other defined feature, in any other aspect or embodiment of the invention.

Furthermore, the present invention is intended to cover apparatus configured to perform any feature described herein in relation to a method and/or a method of using or producing, using or manufacturing any apparatus feature described herein.

### BRIEF DESCRIPTION OF THE DRAWINGS

These and other aspects of the present invention will now be described, by way of example only, with reference to the accompanying drawings, in which:

FIG. 1 is a schematic of an eye imaging system configured for corneal imaging;

FIG. 2 is a schematic of an alternative eye imaging system configured for retinal imaging;

FIG. 3 is a schematic of another alternative eye imaging system configured for retinal imaging;

FIG. 4 is a schematic of a further alternative eye imaging system configured for retinal imaging;

FIG. 5 is a schematic of a handheld eye imaging system configured for retinal imaging;

FIG. 6 is a flowchart of a method of operation of an eye imaging system; and

FIG. 7 is a flowchart of a method of processing captured images.

DETAILED DESCRIPTION OF THE DRAWINGS

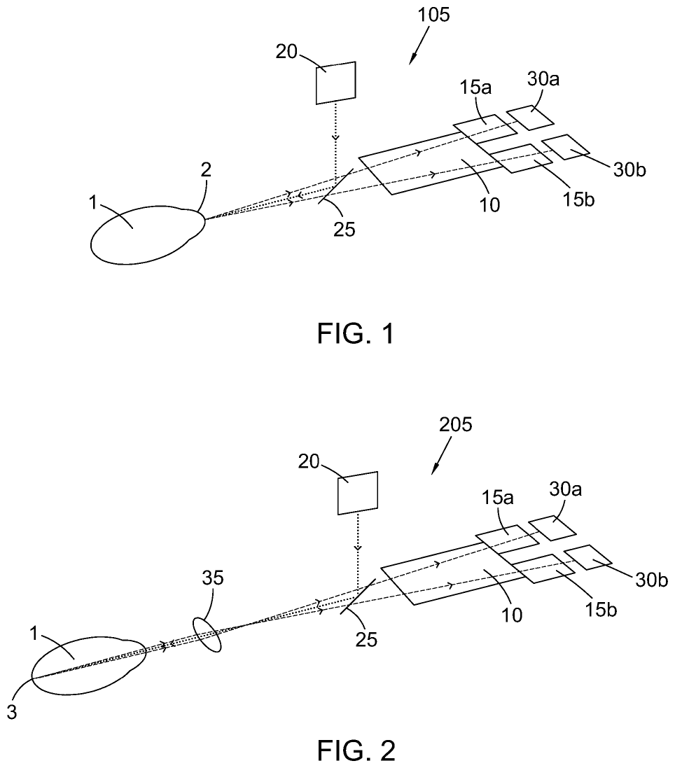

FIG. 1 shows an eye imaging system 105 for imaging a structure of an eye 1, configured to collect images of the cornea of the eye 2 from two different physical locations or at least from two different perspectives. In further examples, the system can be configured to image other structures of the eye such as the retina or optic disc 3, e.g. by the introduction of an indirect lens 35, by varying the distance between the eye and the eye imaging system, and/or the like. Beneficially, with at least some examples described herein, it may be possible to image such structures cost effectively, optionally with simple add-ons to existing equipment, with high spatial resolution, and without causing any discomfort to the patient.

The eye imaging system 105 of FIG. 1 comprises an imaging arrangement, which in this example comprises a slit lamp instrument 10 having a biomicroscope with two apertures 15a, 15b, such as eyepieces or the like, allowing an object to be imaged from two different perspectives e.g. from two different physical locations. The eye imaging system 105 further comprises an image magnification and focus system (not shown) allowing the image viewed through the apertures 15a, 15b to be magnified and focused, thereby facilitating accurate observation of smaller details within the image. The system comprises an illumination device 20 such as a light source, which could be comprised in the imaging arrangement (e.g. as part of the slit lamp arrangement 10) or could be provided separately. Examples of a suitable light source include a slit lamp illuminator, or another light source such as a static image projector, a programmable projector, a display implemented using an LCD, DLP, LCOS, OLED microdisplay, a laser-based device, and/or the like. In addition, although a biomicroscope is described above, a binocular or other suitable device capable of separately providing light reflected from the eye with different perspectives could be used.

In this example, the system further comprises a beam splitter 25 provided in optical pathways between the illumination device 20 and the eye 1 and between the imaging arrangement and the eye 1. The beam splitter 25 reflects a portion, and transmits another portion, of incident light directed towards it. In this example, the beam splitter 25 is configured to reflect light from the illumination device 20 towards the eye 1, and to transmit the light reflected back from structures of the eye to the slit lamp instrument 10, and specifically to the apertures 15a, 15b. The beam splitter 25 may comprise a semi-silvered mirror, prism based beam splitter or other form of device capable of transmitting and reflecting a portion of incident light directed towards it. Although the present example beneficially comprises the beam splitter 25, alternative arrangements that can suitably provide the light from the illumination device 20 to the eye 1 and light reflected from the eye 1 to the imaging arrangement could be used.

Beneficially, the eye imaging system 105 of FIG. 1 comprises two image collection devices 30a, 30b for simultaneously collecting the image viewed through each of the two apertures 15a, 15b of the biomicroscope allowing the images to be stored for further processing. In the example of FIG. 1 the image collection devices 30a, 30b are digital cameras, however, the image collection devices may take other forms.

In the example of FIG. 1 the eye imaging system 105 is configured to collect images of the corneal structure of the eye 2. The illumination device 20 is configured to illuminate the cornea 2 with a patterned or otherwise textured image by reflecting the image through the beam splitter 25 onto the corneal structure of the eye 2. The patterned or otherwise textured image may be produced using a patterned slide positioned between the illumination device 20 and beam splitter 25, or produced directly by the illumination device 20 (e.g. projected by a static image projector), or by other suitable techniques. The patterned or otherwise textured image is then reflected from the corneal structure of the eye 2, from which a portion of the reflected light is transmitted through the beam splitter 25 to the magnification and focus system of the biomicroscope, and therethrough to each of the apertures 15a, 15b of the biomicroscope where it is simultaneously captured from two different physical positions by each of the image collection devices 30a, 30b and stored for further processing, e.g. for forming into 3D images of the structures of the eye 2.

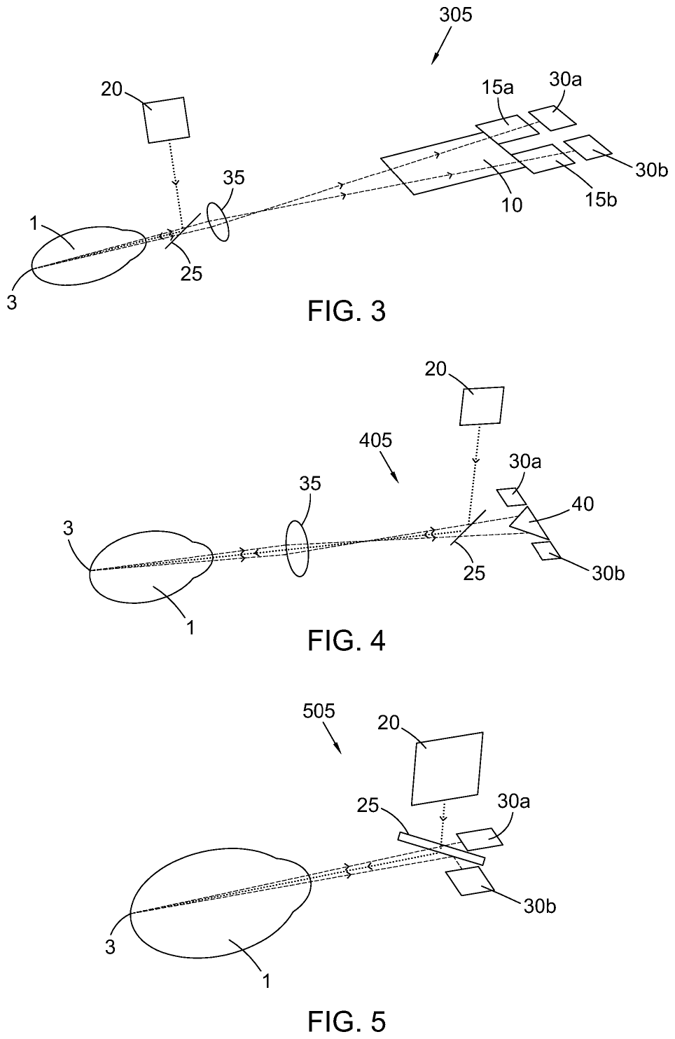

Alternative configurations of the eye imaging system 105 of FIG. 1 configured for imaging the retina of an eye are shown in FIG. 2 and FIG. 3.

In the eye imaging system 205 of FIG. 2, which is similar to the system 105 of FIG. 1, but with an additional indirect lens 35 positioned in an optical path between the eye 1 and beam splitter 25. The indirect lens 35 is configured and positioned to work in conjunction with the eye's optics to conjugate the output of the light source 20 (i.e. the patterned or otherwise textured image) to the retina 3. The patterned or otherwise textured image is then reflected from the retinal structure of the eye 3. The indirect lens 35 then focuses the image to a focus point that is within the focal length of the biomicroscope. A portion of the focused reflected light is transmitted through the beam splitter 25. The use of an indirect lens 35 allows a clear image to be transmitted through the magnification and focus system of the biomicroscope, and through each of the apertures 15a, 15b of the biomicroscope, where it is simultaneously captured from two different physical positions and perspectives by each of the image collection devices 30a, 30b. The images collected by the image collection devices 30a, 30b can then be processed and/or stored for further processing, e.g. for forming into 3D images of the retina 3.

The eye imaging system 305 of FIG. 3 is similar to the system 105 of FIG. 1, but with an additional indirect ophthalmoscopy lens 35 positioned in an optical path between the beam splitter 25 and biomicroscope. The illumination device 20 is positioned at a distance from the eye 1 that advantageously uses the eye's optics to act as a projection lens, projecting the patterned or otherwise textured image from the illumination device 20, reflected by the beam splitter 25, onto the retina 3 through the normal process of vision. Alternatively, a projection lens, or other lens/lens system, is used in combination with the eye's optics to conjugate the image plane of the illumination system to the retina. The patterned or otherwise textured image is then reflected from the retinal structure of the eye 3, from which a portion of the reflected light is transmitted through the beam splitter 25. The indirect ophthalmoscopy lens 35 then focuses the image to a focal point within the focal length of the biomicroscope. This facilitates a clear image being transmitted through the magnification and focus system of the biomicroscope, to each of the apertures 15a, 15b of the biomicroscope. Thereafter, the transmitted light that passes through the apertures 15a, 15b is simultaneously captured from two different physical positions by each of the image collection devices 30a, 30b and stored for further processing, e.g. for formation into 3d images of the retina 3.

FIG. 4 shows an alternative configuration of eye imaging system 405. The eye imaging system 405 of FIG. 4 differs from those of FIGS. 1 to 3 in that, instead of a biomicroscope as in FIGS. 1 to 3, the eye imaging system 405 comprises an image reflection device 40 (e.g. a reflective prism or pair of mirrors). The two image collection devices 30a, 30b are positioned to capture images reflected by the image reflection device 40. This reduces the apparent lateral separation between the image collection devices 30a, 30b to allow them to image overlapping regions of the eye structure 3 simultaneously while having some stereo separation to allow for formation of 3D images from the images collected by the image collection devices 30a, 30b.

In the example of FIG. 4 the eye imaging system 405 is configured to image the retina of an eye 3, however the eye imaging system 405 of FIG. 4 could also be configured to measure other structures of the eye as shown in FIGS. 1 to 3 (e.g. the cornea 2).

FIG. 5 shows an alternative eye imaging system 505 where the apparent lateral separation is reduced using the beam splitter 25, instead of the biomicroscope of FIGS. 1 to 3 or the image reflection device 40 of FIG. 4. In the eye imaging system 505 of FIG. 5, the beam splitter 25 is arranged to reflect the patterned or otherwise textured image from the illumination device 20 towards the structure of the eye 3. However, advantageously, the beam splitter 25 is also arranged to reflect the image reflected from the structure of the eye 3 to one of the two image collection devices 30b, and transmit the image reflected from the structure of the eye to the other of the two image collection devices 30a.

The eye imaging systems of FIGS. 4 and 5 are inherently suitable for miniaturisation, allowing handheld portable configurations. As such the eye imaging systems of FIGS. 4 and 5 are configurable to capture images of the retinal structure of the eye 3 without comprising an indirect lens 35 as the distance between the eye 1 and eye imaging system can be significantly reduced to within the focal length of the eye imaging device. The eye-imaging system 405 of FIG. 4 could be miniaturised by removing the indirect lens 35, facilitating the reduced distance between the eye 1 and eye imaging system 405.

Although various examples are described above, variation to the configuration of the systems described are possible. For example, with the use of different optical elements (e.g. alternative prisms, mirrors or beam splitters) to create the apparent lateral separation of the image capture devices. Furthermore, with small enough image capture devices these optical elements may not be required at all.

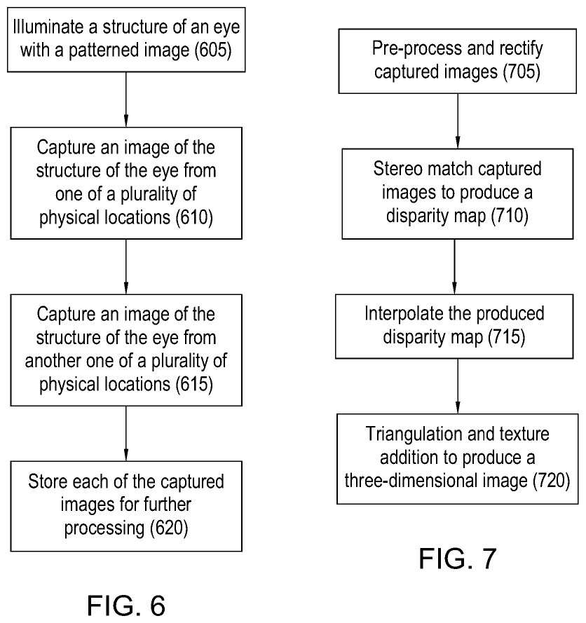

A summary of a method for capturing one or more images of a structure of an eye 1 from a plurality of physical locations and/or perspectives is shown in FIG. 6. This method can be performed by the eye imaging systems of FIGS. 1 to 5 or another suitable eye imaging system.

In step 605 a structure of an eye is illuminated with a patterned or otherwise textured image using an illumination device.

In step 610 an image of the illuminated structure of the eye is captured from one of a plurality of physical locations and/or perspectives.

In step 615 an image of the illuminated structure of the eye is captured from another one of a plurality of physical locations and/or perspectives.

In step 620 each of the captured images are stored for further processing, e.g. for forming 3D images using techniques such as but not limited to that described below in relation to FIG. 7.

It will be appreciated that minimising the opportunity for movement of the eye between capturing an image of the structure of the eye from each of the physical locations and/or perspectives is important. In order to do this, the system beneficially may capture an image from each of the physical locations and/or perspectives substantially simultaneously. Therefore, as far as the eye imaging system used allows, step 610 and step 615 will be performed at substantially the same time. This ensures that any differences identified when processing the images can be substantially attributed to the difference in physical location and/or perspective of the image capture devices and not the movement of the eye. This facilitates an advantageous improvement in the quality of the three dimensional representation of the eye structure that may be produced from the captured images.

A summary of an example method for processing one or more images of a structure of an eye captured from a plurality of physical locations and/or perspectives to produce a three dimensional image of the structure of the eye is shown in FIG. 7. The one or more images may be captured by the eye imaging systems of FIGS. 1 to 5 or another suitable eye imaging system, using the method of FIG. 6 or another suitable method.

In step 705 the captured images are pre-processed and rectified to project the captured images onto a common image plane. Having the images on a common image plane significantly simplifies subsequent processing steps.

In step 710 the rectified images are subjected to a stereo matching process, facilitating identification of the apparent differences between the captured images, to produce a disparity map which contains the depth information. An example of a stereo matching process would involve coarse-to-fine correlation-based patch matching with a left-right consistency check, and including thresholds on patch size and texture to accept matches, however any suitable stereo matching process could be utilised.

In step 715 the disparity map is interpolated to upsample it, permitting a higher resolution 3D image to be obtained. The upsampled disparity map is then subject to triangulation and texture addition in step 720 to produce a three-dimensional image of the structure of the eye.

The method identified in FIG. 7 is merely provided as an example of a process suitable for extracting the necessary depth information from a plurality of images captured from a plurality of physical locations or perspectives, and it will be appreciated that any process, algorithm and/or the like suitable for extracting such information could be implemented. For example, to perform part or all of the process to extract this information, a neural network can be trained to derive intermediate algorithm data, or the full depth information, from two or more images. For example, algorithms akin to what described by Jure Zbontar and Yann LeCun in Journal of Machine Learning Research 17 (2016) 1-32.a can be adapted for this purpose by a person skilled in the art.

Although various examples are described above, variations to the above procedures and apparatus are possible.

For example, references made herein are to imaging a structure of an eye which is under no external load and/or other dynamic factor. However, by adding such a load or and/or other factor statically and/or dynamically to the structure of the eye before and/or during the capture of one or more images, the deformation of the structure of the eye in response to such a load and/or other factor can be quantified. This then allows inference of mechanical properties of the underlying tissues of the structure of the eye.

This technique can be used, for example, in tonometry (the measurement of intraocular pressure by measuring the deformation of the eye surface under a known load) or the application of a retinal tamponade (a chemical or mechanical agent to mechanically affect the retina, e.g. to stop a retinal detachment), or to image otherwise hidden parts of the eye, such as routinely performed in indentation ophthalmoscopy.

In addition, by illuminating the structure of the eye with a dynamic patterned or otherwise textured image, rather than the static image of the approaches described herein, the captured images may allow directionality and rate-dependent phenomena (e.g. fluid flow) to be determined, and/or the sensitivity to be increased e.g. through lock-in detection of image intensity modulation, and/or compensation of image artifacts, and/or the image resolution to be enhanced, and/or structured light imaging procedures to be applied, and/or any other of the image enhancements allowed by image processing of time-variant images.

Method steps of the invention can be performed by one or more programmable processors executing a computer program to perform functions of the invention by operating on input data and generating output. Method steps can also be performed by special purpose logic circuitry, e.g., an FPGA (field programmable gate array) or an ASIC (application-specific integrated circuit) or other customised circuitry. Processors suitable for the execution of a computer program include CPUs and microprocessors, and any one or more processors. Generally, a processor will receive instructions and data from a read-only memory or a random access memory or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including by way of example semiconductor memory devices, e.g. EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in special purpose logic circuitry, such as a manycore vision processing unit, such as e.g. the Intel Movidius Neural Compute Stick, or an inferencing engine, such as e.g. Google Coral or nVidia Jetson Nano.

To provide for interaction with a user, the invention can be implemented on a device having a screen, e.g., a CRT (cathode ray tube), plasma, LED (light emitting diode) or LCD (liquid crystal display), or OLED (organic LED) monitor, or a projector, e.g. a projection system based on LCD, or on a DLP (digital light processing) array, or on a LCOS (liquid crystal on silicon) chip, for displaying information to the user and an input device, e.g., a keyboard, touch screen, a mouse, a trackball, a pedal, and the like by which the user can provide input to the computer. Other kinds of devices can be used, for example, feedback provided to the user can be any form of sensory feedback, e.g., visual feedback, auditory feedback, or tactile feedback; and input from the user can be received in any form, including acoustic, speech, or tactile input. Input can be either voluntary (i.e., the user deliberately provides the input), or involuntary (i.e. a measuring device such as e.g. an accelerometer, or an eye tracker, or a camera measures a user response, e.g. a reflex, that does not require the user needing to make a deliberate action to provide the input, or measures image quality, to trigger the input when the image quality is sufficient for the measurement). Operating on involuntary inputs may be beneficial when the user cannot provide a voluntary input, e.g. because they are too young to understand or obey to instructions, because they are cognitively impaired, etc.

As such, while certain embodiments have been described, these embodiments have been presented by way of example only, and are not intended to limit the scope of the invention. Indeed, the novel methods and systems described herein may be embodied in a variety of other forms. Furthermore, various omissions, substitutions and changes in the form of the methods and systems described herein may be made without departing from the spirit of the invention. The accompanying claims and their equivalents are intended to cover such forms and modifications as would fall within the scope of the invention.

1. An eye imaging system comprising:
an illumination device configured to illuminate a structure of an eye with a patterned or otherwise textured image;
one or more image collection devices wherein;
the one or more image collection devices are arranged to capture one or more images of the structure of the eye from one of a plurality of physical locations and/or from different perspectives.

2. The system of claim 1 further comprising:
an image processing system configured to process the one or more images captured by each of the one or more image collection devices, from each of the physical locations to produce a three dimensional image of the structure of the eye illuminated by the illumination device.

3. The system of claim 1, wherein the patterned image comprises a regular or repeating pattern.

4. The system of claim 1, wherein the patterned image comprises a random or pseudorandom pattern.

5. The system of claim 1, further comprising a beam splitter provided in optical pathways between:
(i) the illumination device and the eye; and
(ii) between the one or more image collection devices and the eye;
the beam splitter being arranged to reflect light from the illumination device towards the eye, and to transmit the light reflected back from the eye to the one or more image collection devices.

6. The system of claim 1, wherein the illumination device comprises an illuminator or other light source.

7. The system of claim 1, wherein the illumination device comprises an image projector.

8. The system of claim 1, wherein the illumination device is configured to have a wavelength which excites a photoluminescent substance applied to the structure of the eye.

9. The system of claim 1, wherein the illumination device is configured to have a wavelength which excites a fluorescent substance applied to the structure of the eye.

10. The system of claim 9 wherein the fluorescent substance comprises fluorescein dye.

11. The system of claim 8, wherein the photoluminescent substance comprises non-fluorescein based photoluminescent materials.

12. The system of claim 8, wherein the one or more image collection devices comprise a light filter configured to filter substantially all of the light, except light at the wavelengths emitted by the fluorescent or photoluminescent substance applied to the structure of the eye, when the structure of the eye is illuminated by the illumination device.

13. The system of claim 1, wherein an indirect ophthalmoscopy lens is positioned between the illumination device and/or the structure of the eye and/or the image collection devices.

14. The system of claim 1, wherein the patterned or otherwise textured image comprises a fine random dot pattern or a set of stripes or a grid.

15. The system of claim 1, wherein the illumination device is configured to illuminate the structure of the eye with the patterned or otherwise textured image that is moving or otherwise changing in time; and the one or more image collection devices are configured to collect multiple images or video of the structure of the eye over time whilst the illumination device projects the moving or otherwise changing image or pattern.

16. A method for capturing one or more images of a structure of an eye using the eye imaging system of claim 1, the method comprising:
illuminating the structure of the eye with a patterned or otherwise textured image using an illumination device; and
using one or more image collection devices:
to capture one or more images of the structure of the eye from one of a plurality of physical locations; and
to capture one or more images of the structure of the eye from another one of the plurality of physical locations and/or from different perspectives.

17. A method for processing one or more images of a structure of an eye, illuminated with a patterned or otherwise textured image by an illumination device, captured by one or more image collection devices, from a plurality of physical locations using an image processing, to produce a three dimensional image of the structure of the eye, wherein
the image processing system is configured to process the one or more images captured by each of the one or more image collection devices, from each of the physical locations to produce a three dimensional image of the structure of the eye illuminated by the illumination device.

18. An eye imaging system comprising:
an illumination device configured to illuminate a structure of an eye with a patterned or otherwise textured image; and
one or more image collection devices arranged to capture one or more images of the structure of the eye; wherein
the illumination device is configured to illuminate the structure of the eye with the patterned or otherwise textured image that is moving or otherwise changing in time; and
one or more image collection devices are configured to collect multiple images or video of the structure of the eye over time whilst the illumination device projects the moving or otherwise changing image or pattern.

19. A method of capturing one or more images of a structure of an eye using an eye imaging system, the eye imaging system comprising an illumination device configured to illuminate a structure of an eye with a patterned or otherwise textured image; and one or more image collection devices arranged to capture one or more images of the structure of the eye, wherein the illumination device if configured to illuminate the structure of the eye with the patterned or otherwise textured image that is moving or otherwise changing in time, and one or more image collection devices are configured to collect multiple images or video of the structure of the eye over time whilst the illumination device projects the moving or otherwise changing image or pattern, and the method comprising:
illuminating the structure of the eye with a moving or otherwise changing patterned or otherwise textured image using the illumination device; and
using one or more image collection devices to capture a video or a plurality of images of the structure of the eye over time whilst the illumination device projects the moving or otherwise changing image or pattern.

20. A non-transient computer readable storage medium comprising a computer program product configured such that, when executed by an eye imaging system, causes the eye imaging system to:
illuminate the structure of the eye with a patterned or otherwise textured image using an illumination device; and
use one or more image collection devices to:
capture one or more images of the structure of the eye from one of a plurality of physical locations; and
capture one or more images of the structure of the eye from another one of the plurality of physical locations and/or from different perspectives.
